(19) United States
(12) Patent Application Publication
(10) Pub. No.: US 2026/0130680 A1
(43) Pub. Date: May 14, 2026

(54) THROMBECTOMY DEVICE INCLUDING CORING RING

(71) Applicant: Bard Peripheral Vascular, Inc., Franklin Lakes, NJ (US)

(72) Inventors: Zachary Willis, Mesa, AZ (US); Austin Jones, Mesa, AZ (US); Ryan Patrick Giudice, Gilbert, AZ (US); Joseph Michael Addante, Phoenix, AZ (US); Greta Raeann Hull, Phoenix, AZ (US)

(21) Appl. No.: 18/941,501

(22) Filed: Nov. 8, 2024

(51) Int. Cl. A61B 17/221; A61B 17/00; A61B 17/32

(52) U.S. Cl. CPC A61B 17/221; A61B 17/00234; A61B 17/32; A61B 2017/00238; A61B 2017/00305; A61B 2017/00477; A61B 2017/00836; A61B 2017/00853; A61B 2017/00862; A61B 2017/00867; A61B 2017/00893; A61B 2017/00942; A61B 2017/2212

(57) ABSTRACT

An example thrombectomy device includes a catheter having a proximal region and a distal region. The thrombectomy device also includes a plurality of atraumatic fingers extending from the proximal region. The atraumatic fingers collectively define an outer periphery of the atraumatic fingers. The thrombectomy device also includes a coring ring attached to the atraumatic fingers such that at least a portion of the coring ring is disposed within the outer periphery of the atraumatic finger. The thrombectomy device also includes a capture bag extending from the distal region of the catheter. The thrombectomy device is configured to switch between a collapsed state and deployed state, wherein the atraumatic fingers, the coring ring, and the capture bag are positioned adjacent to the catheter in the collapsed state and are at least partially spaced from the catheter in the deployed state.

### BACKGROUND

A thrombectomy is a surgical procedure that includes removal of a blood clot from a blood vessel (e.g., artery or vein) to restore blood flow in the blood vessel. The thrombectomy is often performed using catheter-based therapies. Thrombectomy may be an alternative to or performed in conjunction with thrombolytic medications. Thrombectomy may be used to treat any condition involving a thrombus, such as cerebral ischemic strokes, myocardial infarction, and pulmonary embolism.

### SUMMARY

Embodiments disclosed herein are related to thrombectomy devices, thrombectomy systems including the same, and methods of making and using the same. In an embodiment, a thrombectomy device is disclosed. The thrombectomy device includes a catheter having a proximal region and a distal region and a plurality of atraumatic fingers extending from the proximal region. The plurality of atraumatic fingers collectively define an outer periphery of the plurality of atraumatic fingers. The thrombectomy device also includes a coring ring attached to or integrally formed with at least some of the plurality of atraumatic fingers. At least a portion of the coring ring is positioned within the outer periphery of the plurality of atraumatic fingers. The thrombectomy device further includes a capture bag extending from the distal region. The thrombectomy device is switchable between a compact state and a deployed state. The plurality of atraumatic fingers, the coring ring, and the capture bag are adjacent to the catheter when the thrombectomy device is in the compact state and at least partially spaced from the catheter when the thrombectomy device is in the deployed state.

In an embodiment, a thrombectomy system is disclosed. The thrombectomy system includes a thrombectomy device. The thrombectomy device includes a catheter having a proximal region and a distal region and a plurality of atraumatic fingers extending from the proximal region. The plurality of atraumatic fingers collectively define an outer periphery of the plurality of atraumatic fingers. The thrombectomy device also includes a coring ring attached to or integrally formed with at least some of the plurality of atraumatic fingers. At least a portion of the coring ring is positioned within the outer periphery of the plurality of atraumatic fingers. The thrombectomy device further includes a capture bag extending from the distal region. The thrombectomy device is switchable between a compact state and a deployed state. The plurality of atraumatic fingers, the coring ring, and the capture bag are adjacent to the catheter when the thrombectomy device is in the compact state and at least partially spaced from the catheter when the thrombectomy device is in the deployed state. The thrombectomy system also includes an external containment sheath moveably disposed on at least partially of the thrombectomy device. The thrombectomy device is in the collapsed state when the external containment sheath is disposed over the plurality of atraumatic fingers, the coring ring, and the capture bag. Displacing the thrombectomy device distally out of the external containment sheath a sufficient distance switches the thrombectomy device from the collapsed state to the deployed state.

In an embodiment, a method of using a thrombectomy device is disclosed. The method includes advancing the thrombectomy device in a collapsed state through a thrombus such that a plurality of atraumatic fingers of the thrombectomy device is positioned distal to the thrombus. The thrombectomy device includes a catheter having a proximal region and a distal region and the plurality of atraumatic fingers extending from the proximal region. The plurality of atraumatic fingers collectively define an outer periphery of the plurality of atraumatic fingers. The thrombectomy device also includes a coring ring attached to or integrally formed with at least some of the plurality of atraumatic fingers. At least a portion of the coring ring is positioned within the outer periphery of the plurality of atraumatic fingers. The thrombectomy device also includes a capture bag extending from the distal region. The plurality of atraumatic fingers, the coring ring, and the capture bag are adjacent to the catheter when in the collapsed state. The method also includes moving an external containment sheath proximally for a sufficient distance to switch the thrombectomy device from the collapsed state to a deployed state. The coring ring, and the capture bag are at least partially spaced from the catheter when the thrombectomy device is in the deployed state. The method further includes advancing the thrombectomy device proximally to cut the thrombus from a wall of a blood vessel and dispose the cut thrombus in the capture bag.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a side view of a portion of a thrombectomy device in the deployed state, according to an embodiment.

FIG. 1B is a cross-sectional view of the thrombectomy device taken along plane 1B-1B.

FIG. 2 is a side view of a thrombectomy device, according to an embodiment.

FIGS. 3A-3C are a side view of a thrombectomy system including a thrombectomy device and configured to switch the thrombectomy device from a collapsed state to a deployed state, according to an embodiment.

FIGS. 4A-4D illustrate a method of using a thrombectomy system, according to an embodiment.

### DETAILED DESCRIPTION

Embodiments disclosed herein are related to thrombectomy devices, thrombectomy systems including the same, and methods of making and using the same. An example thrombectomy device includes a catheter having a proximal region and a distal region. The thrombectomy device also includes a plurality of atraumatic fingers extending from the proximal region. The atraumatic fingers collectively define an outer periphery of the atraumatic fingers. The thrombectomy device also includes a coring ring attached to the atraumatic fingers such that at least a portion of the coring ring is disposed within the outer periphery of the atraumatic finger. The thrombectomy device also includes a capture bag extending from the distal region of the catheter. The thrombectomy device is configured to switch between a collapsed state and deployed state, wherein the atraumatic fingers, the coring ring, and the capture bag are positioned adjacent to the catheter in the collapsed state and are at least partially spaced from the catheter in the deployed state.

During use, the thrombectomy device, while in the collapsed state, may extend through a thrombus such that the proximal region of the catheter is positioned on a distal side of the thrombus. It is noted that, as used herein, proximal and distal means a region or side that is closer or further, respectively, to an incision that allows the thrombectomy device to reach a region of interest (e.g., thrombus) along the path taken by the thrombectomy device from the incision to the region of interest. Similarly, proximal and distal directions refer to directions that extend towards or away from, respectively, the incision along the path taken by the thrombectomy device. After extending the thrombectomy device through the thrombus, the thrombectomy device may be switched from the collapsed state to a deployed state. The thrombectomy device may switch from the collapsed state to the deployed state by moving an external containment sheath proximally such that the external containment sheath does not cover the atraumatic fingers, the coring ring, and the capture bag. The thrombectomy device may then be advanced in the blood vessel in a proximal direction. Such advancement of the thrombectomy device may cause the coring ring to cut the thrombus from the walls of the blood vessel and the cut thrombus to be disposed in the capture bag. The thrombectomy device, with the thrombus in the capture bag, may then be removed from the blood vessel thereby removing the thrombus from the blood vessel.

The thrombectomy device is an improvement over conventional thrombectomy devices. The walls of the blood vessel and the thrombus often exhibit a similar density, are strongly attached to each other, and/or are otherwise difficult to remove from the blood vessels. As such, advancing conventional thrombectomy devices through the thrombus often causes the conventional thrombectomy devices to cut, scrape, or otherwise damage the walls of the blood vessel which can lead to re-thrombosis and lake of a clinical benefit. In an example, some conventional thrombectomy devices includes longitudinally-extending supports and a cutting element disposed on the outside of the longitudinally-extending members. Advancing such a conventional thrombectomy device through the thrombus causes the cutting element to scrape along and cut the walls of the blood vessels. In an example, some conventional thrombectomy devices rely on rotation of one or more components of the thrombectomy devices to separate the thrombus from the blood vessel. However, the rotating components of the conventional thrombectomy devices may also rub, scrape, or otherwise damage the walls of the blood vessel. The rotating components can also lyse blood cells and make them unsuitable for re-infusion comment in these types of procedures which, in turn, may lead to increased blood loss during the procedure. The conventional thrombectomy devices including the rotating components (and other thrombectomy devices) may also require an aspiration function for emboli protection which may also increase blood loss. Further, the rotation of such conventional thrombectomy devices includes several mechanical connections that increase the complexity of the conventional thrombectomy devices and increases the likelihood that the conventional thrombectomy devices fail during use.

The thrombectomy devices disclosed herein are an improvement over such conventional thrombectomy devices. For example, the atraumatic fingers do not include a cutting edge and, instead, includes smooth surfaces with rounded edges that can contact the walls of the blood vessels. As such, the atraumatic fingers are unlikely to damage the walls of the blood vessel as the thrombectomy devices disclosed herein are advanced through a thrombus. As previously discussed, at least a portion of the coring ring is disposed within the outer periphery defined by the atraumatic fingers. As such, the atraumatic fingers isolate at least a portion (e.g., all) of the coring ring from the walls of the blood vessel and prevent or at least inhibit the coring ring from damaging the walls of the blood vessel. Further, the thrombectomy devices disclosed herein are able to separate the thrombus from the walls of the blood vessel without requiring any of the components of the thrombectomy devices disclosed herein to rotate relative to each other thereby making the thrombectomy devices less likely to fail (though, it is noted, at least some of the components of the thrombectomy devices may rotate relative to the walls of the blood vessel).

It is noted that other improvements of the thrombectomy devices disclosed herein compared to conventional thrombectomy devices are disclosed below.

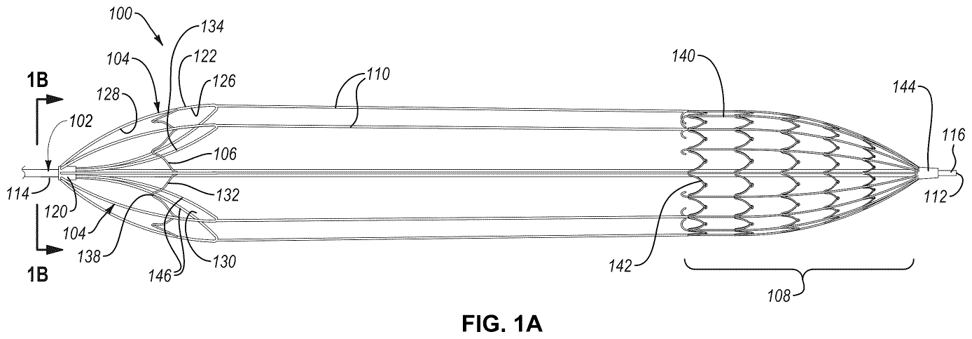

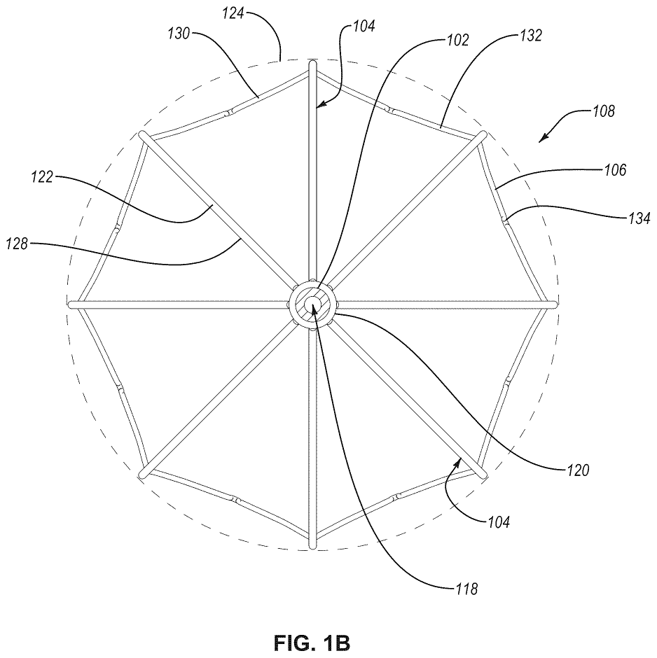

FIG. 1A is a side view of a portion of a thrombectomy device 100 in the deployed state, according to an embodiment. FIG. 1B is a cross-sectional view of the thrombectomy device 100 taken along plane 1B-1B. The thrombectomy device 100 includes a catheter 102 and a plurality of atraumatic fingers 104 extending from the catheter 102. The thrombectomy device 100 also includes a coring ring 106. The thrombectomy device 100 further includes a capture bag 108 distally spaced from the atraumatic fingers 104 and the coring ring 106. The thrombectomy device 100 may optionally include a plurality of linkages 110 extending from the fingers 104 or the coring ring 106 to the capture bag 108.

The catheter 102 is an elongated element that extends from a proximal end (not shown) that, during use, may be outside of a body to a distal end 112. The catheter 102 provides a support body to support the other components (e.g., the atraumatic fingers 104, the coring ring 106, the capture bag 108, etc.) of the thrombectomy device 100. The catheter 102 also provides a means to move the components in a blood vessel by, for instance, pushing or pulling on the portions of the catheter 102 that are outside of the body. For example, the catheter 102 provides a means to move the other components of the thrombectomy device 100 through a thrombus and then advance the other components proximally such that the coring ring 106 separates at least a portion of the thrombus from the walls of the blood vessel.

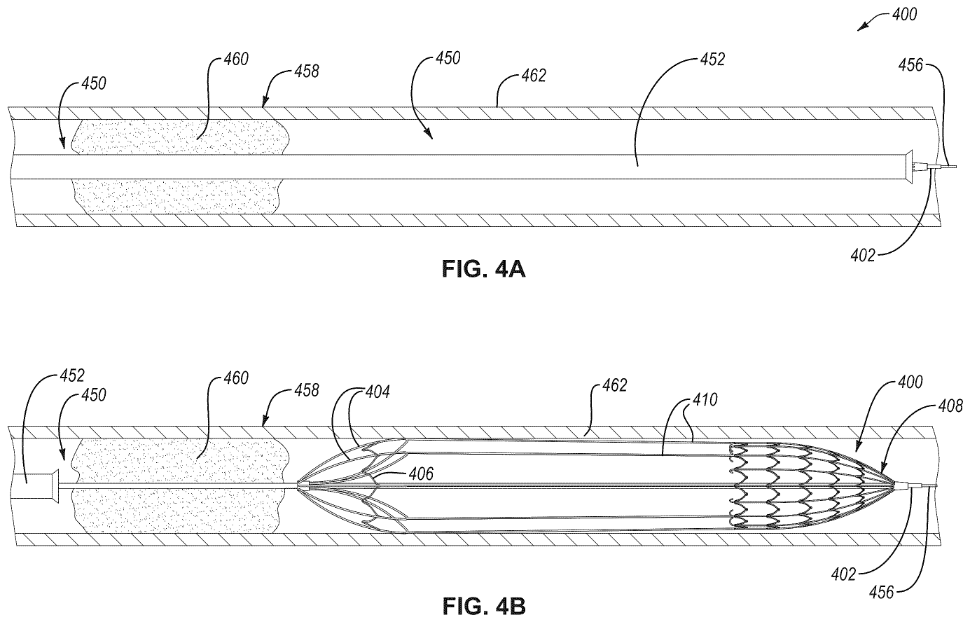

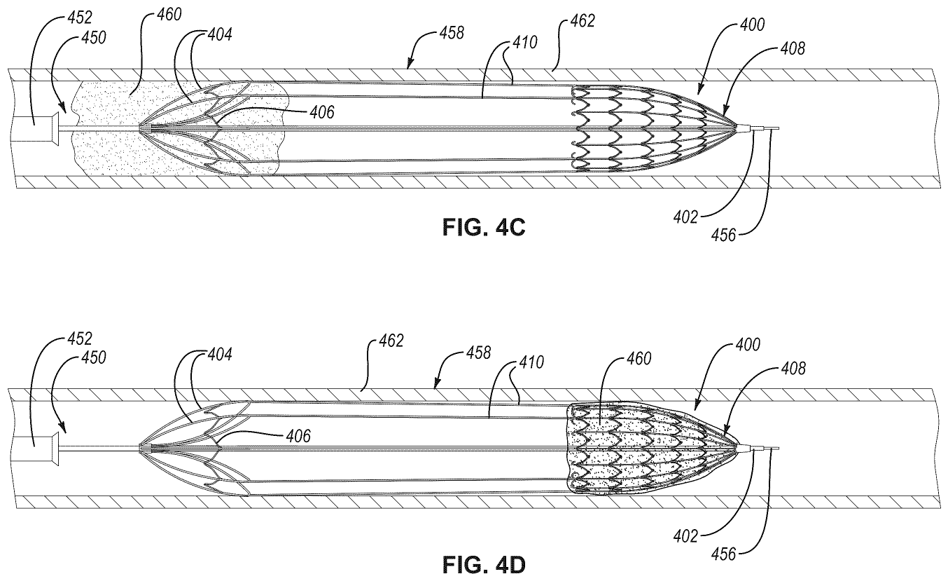

The catheter 102 includes a proximal region 114 and a distal region 116. The proximal region 114 of the catheter 102 is the portion of the catheter 102 that the atraumatic fingers 104 are attached to and extend from. The distal region 116 of the catheter 102 is the portion of the catheter 102 that the capture bag 108 is attached to. The proximal region 114 is generally spaced from the proximal end of the catheter 102 by a significant distance (e.g., about 25 cm to about 200 cm, about 50 cm to about 150 cm, or about 70 cm to about 80 cm). The distal region 116 may also be spaced significantly from the distal end 112 of the catheter 102 though, generally, the distal region 116 is at or near the distal end 112. The catheter 102 may also define a hollow cavity 118 that is configured to receive a guidewire (e.g., guidewire 456 of FIGS. 4A-4D).

In an embodiment, the catheter 102 may be the same as or substantially to the catheters of conventional thrombectomy devices. In an embodiment, the catheter 102 may include at least one of a poly(ether-block-amide), a polyamide, a nylon, a polyimide, other thermoplastic elastomers, a reinforced polymer, any other suitable material, or combinations thereof. The catheter 102 may be distinct from a guidewire (e.g., guidewire 456 illustrated in FIGS. 4A-4D) used to position the catheter 102 in an individual.

As previously discussed, the atraumatic fingers 104 extend distally from the proximal region 114 of the catheter 102. For example, the atraumatic fingers 104 may extend in a direction that is oblique relative to the longitudinal axis of the catheter (e.g., extending in a direction that is both radially outwardly from the catheter 102 and towards the distal region 116). The atraumatic fingers 104 are configured to ride along an inner surface of a wall of a blood vessel. As such, the atraumatic fingers 104 act as a buffer or barrier between the wall of the blood vessel and any cutting or scraping performed by at least some other components of the thrombectomy device 100 (e.g., the coring ring 106). As will be discussed in more detail below, the atraumatic fingers 104 may be attached to the coring ring 106. As such, actuation of the atraumatic fingers 104 that switching the atraumatic fingers 104 from the collapsed state to the deployed state (as will be discussed in more detail below) may cause the atraumatic fingers 104 to also actuate the coring ring 106 from the collapsed state to the deployed state.

The atraumatic fingers 104 may be attached to the catheter 102 using any suitable technique. In an example, as shown, the thrombectomy device 100 includes a proximal sleeve 120 disposed over the catheter 102 and the atraumatic fingers 104 may be integrally formed (e.g., exhibit single piece construction) with or attached to the proximal sleeve 120 (e.g., via a weld, melting one or more of the sleeve 120 or atraumatic fingers 104, etc.). The proximal sleeve 120 may be attached to the catheter 102 using any suitable technique, such as via an interference fit, an adhesive, a weld, or any other suitable technique. In an example, the atraumatic fingers 104 may be directly attached to the catheter 102 via a weld, melting the catheter 102 around the atraumatic fingers 104, using an adhesive, or any other suitable technique.

The atraumatic fingers 104 includes an exterior surface 122. The exterior surface 122 is the surface of the atraumatic fingers 104 that is furthest spaced from the catheter 102 and/or is configured to abut the wall of the blood vessel during use. The exterior surfaces 122 of the atraumatic fingers 104 collective defines an outer periphery 124 (shown with a dashed line) taken in a plane that is perpendicular to the longitudinal axis of the catheter 102. The outer periphery 124 represents the area that may be protected or substantially protected by the atraumatic fingers 104. For example, the thrombectomy device 100 may be configured to prevent or at least inhibit damage to any structure (e.g., blood vessel wall) that is not within the outer periphery 124 but may be configured to cut or otherwise damage at least some of the structures (e.g., thrombus) that are within the outer periphery 124.

The outer periphery 124 may be a rounded shape (as shown) or may be straight lines extending between adjacent exterior surfaces 122. For example, the outer periphery 124 may be a generally circular shape, a generally oval shape, or a generally angular shape having as many sides as there are atraumatic fingers 104 (e.g., eight atraumatic fingers 104 may cause the outer periphery 124 to exhibit a generally octagonal shape). The shape of the outer periphery 124 may be selected based on the cross-sectional shape of the blood vessel or the blood vessel restricting further expansion of the atraumatic fingers 104. The maximum lateral dimension of the outer periphery 124 may increase with increasing distance from the proximal region 114 along at least a portion of a length of the atraumatic fingers 104 measured in a direction extending parallel to the longitudinal axis of the catheter 102. The maximum lateral dimension of the outer periphery 124 of the atraumatic fingers 106 may be about 10 mm to about 25 mm, such as about 12 mm to about 20 mm.

The thrombectomy device 100 may include any suitable number of atraumatic fingers 104. For example, the thrombectomy device 100 may include 3, 4, 5, 6, 7, 8, 9, 10, 11, 12, 13, 14, 15, 16, or more than 16 atraumatic fingers 104. Generally, increasing the number of atraumatic fingers 104 better defines the outer periphery 124 (i.e., the boundary between which the atraumatic fingers 104 can and cannot prevent such damage). Increasing the number of atraumatic fingers 104 may also better support the walls of the blood vessel such that portions of walls of the blood vessel do not sag between adjacent atraumatic fingers 104 and through the outer periphery 124. However, it may be difficult to cut the portions of the thrombus that comes into direct contact with the atraumatic fingers 104 with the coring ring 106 since the atraumatic fingers 104 are between these portions of the thrombus and the coring ring 106. Instead, such portion of the thrombus may be cut using shear forces applied by the atraumatic fingers 104 which may result in tearing of the thrombus instead of cutting the thrombus and may make advancing the coring ring 106 into the thrombus more difficult. As such, the number of atraumatic fingers 104 may be limited by the desire to limit the number of portions of the thrombus that the coring ring 106 cannot easily directly cut.

As previously discussed, the exterior surfaces 122 of the atraumatic fingers 104 may make direct contact the walls of the blood vessel during use. As such, the exterior surfaces 122 may be configured to prevent or at least inhibit damage or irritation to the walls of the blood vessels as the thrombectomy device 100 moves relative to the blood vessel when in the deployed state. For example, the exterior surfaces 122 may exhibit a relatively smooth (e.g., polished) surface finish, such as a surface finish (in Ra) of about 25 μm or less, about 15 μm or less, about 10 μm or less, about 5 μm or less, about 2.5 μm or less, about 1 μm or less, about 0.5 μm or less, about 0.1 μm or less, or about 0.05 μm or less. Such surface finishes may prevent rough portions of the exterior surfaces 122 abrading the walls of the blood vessel, for example, due to friction between the exterior surfaces 112 and the walls of the blood vessels. In an embodiment, the exterior surfaces 122 may exhibit relatively gentle curves which may prevent or inhibit the atraumatic fingers 104 inadvertently grabbing or tugging on the walls of the blood vessels. In an embodiment, the interior surfaces 126 opposite the exterior surfaces 122 and the lateral surfaces 128 extending between the exterior surfaces 122 and the interior surfaces 126 may not exhibit such surface finishes or gentle curves if the atraumatic fingers 104 do not rotate relative to the walls of the blood vessel during use since, without rotation, the interior and lateral surfaces 126, 128 are unlikely to directly contact the walls of the blood vessel. That said, the interior surfaces 126 and the lateral surfaces 128 may exhibit any of the above surface finishes and/or exhibit gentle curves.

The atraumatic fingers 104 may be formed from any suitable material. In an example, the atraumatic fingers 104 may include a nickel-titanium superelastic material which may allow the atraumatic fingers 104 to switch from the collapsed state to the deployed state without using a spring. In an example, the atraumatic fingers 104 may include steel, titanium, another biocompatible metal, a biocompatible polymer, a biocompatible composite, or combinations of any of the materials disclosed herein.

As previously discussed, the thrombectomy device 100 includes a coring ring 106. The coring ring 106 is configured to cut at least a portion of any structure that is within the outer periphery 124. For example, during use the coring ring 106 may cut at least a portion of the thrombus that is within the outer periphery 124 thereby separating at least a portion of the thrombus from the walls of the blood vessel and allowing subsequent removal of the cut thrombus from the blood vessel. The coring ring 106 may be prevent or at least partially inhibited from cutting or otherwise damaging any structure that is outside of the outer periphery 124.

The coring ring 106 may include a cutting surface that is configured to cut the thrombus or any other structure that is within the outer periphery 124. In an embodiment, as shown, the coring ring 106 includes a cutting edge 130 (i.e., sharpened edge) on a proximal side of the coring ring 106. The cutting edge 130 may be formed by at least one chamfered surface or be otherwise sharpened. In an example, as shown, the cutting edge 130 is spaced from an exterior surface 132 of the coring ring 106. In such an example, the cutting edge 130 may not cut or scrape against the wall of the blood vessel that lightly contacts the exterior surface 132. In an example, as shown, the cutting edge 130 extends along at least a portion (e.g., an entirety) of a circumference of the coring ring 106. In an example, the cutting edge 130 is only formed in the proximally forwardmost portions of the coring ring 106 (e.g., the peaks 134). In such an example, the cutting edge 130 may cut the portions of the thrombus that contact the cutting edge 130 and the other portions of the coring ring 106 may apply a shearing force to the thrombus that promotes tearing of the thrombus between the cut portions of the thrombus. In an example, the cutting edge 130 may not be formed on portions of the coring ring 106 that are closest to the outer periphery 124 or extend past the outer periphery 124. Instead, the portions of the coring ring 106 that are closest to the outer periphery 124 or extend past the outer periphery 124 may include smooth and/or rounded edges to minimize damage caused by this portion of the coring ring 106 to the walls of the blood vessel. It is noted that these portions of the coring ring 106 that include smooth and/or rounded edges may instead apply a shear force to the thrombus that promotes tearing of the thrombus.

In an embodiment, as shown, the coring ring 106 may exhibit a generally zigzag shape. For example, the coring ring 106 may have peaks 134 that form the proximally facing portions of the coring ring 106 and valleys 136 between the peaks 134 that form the distally facing portions of the coring ring 106. The zigzag structure of the coring ring 106 facilitates collapsing of the coring ring 106 when the thrombectomy device 100 is in the collapsed state. In particular, the zigzag shape allows the coring ring 106 to fold at the peaks 134 and the valleys 136 until the diameter of the coring ring 106 measured perpendicular to the longitudinal axis of the catheter 102 matches or is similar to the diameter of the catheter 102. In an embodiment, the valleys 136 may be attached or integrally formed with the atraumatic fingers 104. In such an embodiment, the peaks 134 are free to engage with and cut the thrombus. The peaks 134 better facilitate cutting the thrombus than the valleys 136 since the peaks 134 exhibit a pointed-like structure that may more easily penetrate the thrombus. It is noted that the coring ring 106 may exhibit other collapsible shapes, as known in the art. The peaks 134 and the valleys 136 of the coring ring 106 may form a serration feature that better engages and separates the thrombosis from the vessel walls than a coring ring 106 that does not include the peaks 134 and the valleys 136.

In an embodiment, as shown, the coring ring 106 may exhibit a generally circular cross-sectional shape taken along a plane extending perpendicular to the longitudinal axis of the catheter 102. The generally circular cross-sectional shape of the coring ring 106 decreases the likelihood that the coring ring 106 comes in contact with the walls of the blood vessel during operation. However, the generally circular cross-sectional shape of the coring ring 106 may cause the coring ring 106 to be spaced from the outer periphery 124. Portions of the thrombus that are between the coring ring 106 and the outer periphery 124 may not be cut or otherwise separated from the walls of the blood vessels. That said, the volume of the thrombus that may remain attached to the walls of the blood vessel may be sufficiently small that thrombolytic drugs may be able to effectively remove the remaining portions of the thrombus, depending on the thrombus's chronicity. In an embodiment, the coring ring 106 may exhibit an angular shape having as many sides as the thrombectomy device 100 includes atraumatic fingers 104. However, in such an embodiment, the coring ring 106 may have many of the same problems as the generally circular coring ring 106. In an embodiment, portions of the coring ring 106 may extend radially outwardly from other portions of the coring ring 106 such that some portions of the coring ring 106 are closer the outer periphery 124 than other portions of the coring ring 106 or extends past the outer periphery 124. In such an embodiment, the coring ring 106 may exhibit a generally star-like cross-sectional shape or other similar cross-sectional shape taken along a plane extending perpendicular to the longitudinal axis of the catheter 102. While the portions of the coring ring 106 extending radially outwardly from other portions of the coring ring 106 may increase the percentage of the thrombus that is cut and separated from the walls of the blood vessel, such portions of the coring ring 106 may also increase the likelihood that the coring ring 106 inadvertently cut or otherwise damage the walls of the blood vessel. In a particular example, as previously discussed, the portions of the coring ring 106 that extend radially outwardly from other portions of the coring ring 106 may not include the cutting edge 130.

The coring ring 106 is attached to or integrally formed (i.e., exhibits single piece construction) with the atraumatic fingers 104. When the coring ring 106 is attached to the atraumatic fingers 104, the coring ring 106 may be attached to the atraumatic fingers 104 using any suitable technique, such as welding or using an adhesive. Attaching the coring ring 106 to the atraumatic fingers 104 may facilitate the manufacturing and operation of the thrombectomy device 100. For instance, the atraumatic fingers 104 may be formed from a nickel-titanium superelastic alloy (e.g., nitinol) or other superelastic material which allows the atraumatic fingers 104 to deploy when an external compressive force is removed (e.g., the external container sheath is removed from the atraumatic fingers 104 and the thrombectomy device 100 can switch from the collapsed state to the deployed state). However, it is difficult to form the cutting edge 130 in a nickel-titanium superelastic alloy. Attaching the coring ring 106 to the atraumatic fingers 104 allows the coring ring 106 to be formed from a material on which it is easier to form the cutting edge 130 than a nickel-titanium superelastic alloy, such as stainless steel, titanium, or another metal.

The coring ring 106 may be attached to or integrally formed with any surface of the atraumatic fingers 104. In an example, the coring ring 106 is attached to or integrally formed with the interior surfaces 126 of the atraumatic fingers 104. In such an example, at least a portion of the coring ring 106 may be spaced from the outer periphery 124 by the thickness of the atraumatic fingers 104 and are unlikely to contact the walls of the blood vessels during use. In an example, the coring ring 106 may be attached to or integrally formed with at least a portion of the lateral surfaces 128 of the atraumatic fingers 104 which allows the coring ring 106 to be positioned closer to the outer periphery 124 and decreases the volume of the thrombus that is not removed by the coring ring 106.

The coring ring 106 may include one or more bending regions 138 (e.g., slots, perforations, thinned regions, etc.) extending at least partially (e.g., completely) therethrough. The bending regions 138 may be formed in portions of the coring ring 106 that are likely to bend, or it is desirable to bend when switching the thrombectomy device 100 from the deployed state to the collapsed state. For example, the bending regions 138 may be formed in the peaks 134 and the valleys 136 of the coring ring 106. The bending regions 138 weaken these portions of the coring ring 106 thereby facilitating bending of these portions of the coring ring 106. In an embodiment, as illustrated, the bending regions 138 include slots extend inwardly from an edge of the coring ring 106 such that the slots are only partially defined by the coring ring 106. These slots may extend inwardly from a concave surface of the coring ring 106 and/or a surface of the coring ring 106 that does not form the cutting edge 130. In an embodiment, the bending regions 138 may include perforations that are completely defined by the coring ring 106.

As previously discussed, the thrombectomy device 100 includes a capture bag 108. The capture bag 108 is distally spaced from the coring ring 106. For example, the capture bag 108 may be spaced from the coring ring 106 by about 25 mm to about 200 mm, such as about 50 mm to about 150 mm or about 50 mm to about 100 mm. The capture bag 108 is also attached to the distal region 116 of the catheter 102. The capture bag 108 is configured to receive and hold the portions of the thrombus that are cut and separated from the walls of the blood vessel. The capture bag 108 is also configured to prevent or at least inhibit pieces of the thrombus from flowing therethrough such that these pieces of the thrombus are not removed from the blood vessel. As such, the capture bag 108 may define gaps between the components thereof that are relatively small compared to other gaps formed between other components of the thrombectomy device 100. That said, the capture bag 108 may still define gaps to allow blood or other fluids to flow therethrough as the thrombectomy device 100 is moved in the blood vessel. The capture bag 108 may exhibit a maximum length measured parallel to the catheter 102 that is about 50 mm to about 300 mm, such as about 100 mm to about 200 mm.

In an embodiment, the capture bag 108 may include one or more longitudinally extending elements 140 and one or more circumferentially extending elements 142. At least a portion of the longitudinally extending elements 140 may generally extend in a direction that is parallel to the longitudinal axis of the catheter 102 though, it is noted, some portions (e.g., portions near the distal region 116) of the longitudinally extending elements 140 may also extend radially. It is noted that the linkages 110 may be integrally formed with at least some of the longitudinally extending elements 140. The circumferentially extending elements 142 may extend between the longitudinally extending elements 140. The circumferentially extending elements 142 may, as a whole, extend in a generally circumferential direction though local portions of the circumferentially extending elements 142 may extend in a non-circumferential direction. For example, as shown, the circumferentially extending elements 142 may exhibit a zigzag-like structure though, as a whole, the circumferentially extending elements 142 extend in a circumferential direction. The zigzag-like structure of the circumferentially extending elements 142 may facilitate collapsing the capture bag 108. It is noted that the capture bag 108 may include other elements other than or in addition to the longitudinally and circumferentially extending elements 140, 142, such as helically extending elements. At least some of these other elements may be integrally formed with the linkages 110. In an embodiment, the capture bag 108 may exhibit a structure used in conventional thrombectomy capture bags, such as the same structure as the capture bag of the ClotTriever® Thrombectomy System.

In an embodiment, the terminal ends of the elements (e.g., the longitudinally extending elements 140) that are on a proximal side of the capture bag 108 and do not form part of the linkages 110 may be curved downwardly (e.g., towards the catheter 102). These downwardly curved terminal ends of the elements may prevent or at least inhibit the terminal ends of the elements from catching against the walls of the blood vessel as the thrombectomy device 100 moves in the blood vessel.

At least some of the elements of the capture bag 108 (e.g., the longitudinally extending elements 140) may be attached to distal region 116 of the catheter 102 using any suitable technique. In an example, as shown, the thrombectomy device 100 includes a distal sleeve 144 disposed over the catheter 102 and the capture bag 108 may be integrally formed (e.g., exhibit single piece construction) with or attached to the distal sleeve 144 (e.g., via a weld, melting one or more of the sleeve 120 or capture bag 108, etc.). The distal sleeve 144 may be attached to the catheter 102 using any suitable technique, such as via an interference fit, an adhesive, a weld, or any other suitable technique. In an example, the capture bag 108 may be directly attached to the catheter 102 via a weld, melting the catheter 102 around the capture bag 108, using an adhesive, or any other suitable technique.

As previously discussed, the thrombectomy device 100 may include a plurality of linkages 110. The linkages 110 may connect the capture bag 108 with at least one of the atraumatic fingers 104 or the coring ring 106. As such, the linkages 110 provide structural support to the thrombectomy device 100. The linkages 110 may also direct the thrombus cut by the coring ring 106 towards the capture bag 108 such that the thrombus does not overextend the capture bag 108 which may inhibit advancement of the thrombectomy device 100 in the blood vessel and may allow portions of the thrombus to remain in the blood vessel. The linkages 110 may also preclude the need to downwardly curve the terminal ends of the elements of the capture bag 108 that are on the proximal side of the capture bag 108.

The linkages 110 may extend generally parallel to a longitudinal axis of the catheter 102, as shown. The linkages 110 may exhibit a length, measured between the atraumatic fingers 104 or the coring ring 106 to the capture bag 108, that is about 25 mm to about 200 mm, such as about 50 mm to about 150 mm or about 50 mm to about 100 mm. The generally parallel orientation of the linkages 110 may facilitate movement of the thrombus towards the capture bag 108 since the orientation of the linkages 110 may be aligned with the direction that the thrombus moves after being cut and before being received in the capture bag 108. That said, the linkages 110 may exhibit other orientations. For example, the linkages 110 may extend in a generally helical orientation relative to the longitudinal axis of the catheter 102. The atraumatic fingers 104 may cut the thrombus in multiple pieces as the atraumatic fingers 104 move through the thrombus. The cuts formed in the thrombus by the atraumatic fingers 104 may extend generally aligned with the longitudinal axis of the catheter 102. The helical arrangement of the linkages 110 may prevent or at least inhibit multiple pieces of the thrombus from passing through the gaps of the linkages 110.

In an embodiment, as shown, the linkages 110 are integrally formed with at least one of the atraumatic fingers 104 or at least some of the elements (e.g., at least some of the longitudinally extending elements 140) of the capture bag 108. Integrally forming the linkages 110 with the atraumatic fingers 104 may simplify manufacturing of the thrombectomy device 100 since the linkages 110 do not need to be attached to the atraumatic fingers 104 and/or the elements of the capture bag 108. In an embodiment, the linkages 110 may be distinct from and attached to at least one of the atraumatic fingers 104 or at least some of the elements of the capture bag 108.

The linkages 110 may be distinguishable from the atraumatic fingers 104 and the capture bag 108 for one or more reasons. In an example, the linkages 110 are distinguishable from the atraumatic fingers 104 because the linkages 110 are located distally from the coring ring 106 such that the linkages 110 provide little to no protection to the walls of the blood vessels from the coring ring 106. In an example, the linkages 110 are distinguishable from the capture bag 108 because the linkages 110 define gaps therebetween that are significantly larger than the gaps defined by the capture bag 108 since the linkages 110 are not configured to hold the thrombus but instead merely facilitate moving the thrombus towards the capture bag 108 that is configured to hold the thrombus. In an example, as shown, the thrombectomy device 100 may not include circumferentially extending elements between the linkages 110. That said, in some examples, the thrombectomy device 100 may include circumferentially extending elements between the linkages 110. These circumferentially extending elements between the linkages 110 may prevent or at least inhibit some of the cut thrombus from passing through the gaps of the linkages 110 and may provide additional structural support between the linkages 110. The circumferentially extending elements extending between the linkages 110 may be further spaced from each other than the circumferential extending elements 142 of the capture bag 108 since, as previously discussed, the linkages 110 are not configured to hold the thrombus. In an embodiment, linkages 110 may be distinguishable from the atraumatic fingers 104 and the capture bag 108 because the linkages 110 may extend generally parallel and/or not extend at an angle that would intersect with the longitudinal axis of the catheter 102 since, unlike the atraumatic fingers 104 and/or at least a portion of the capture bag 108, the linkages 110 may not be directly attached to the catheter 102.

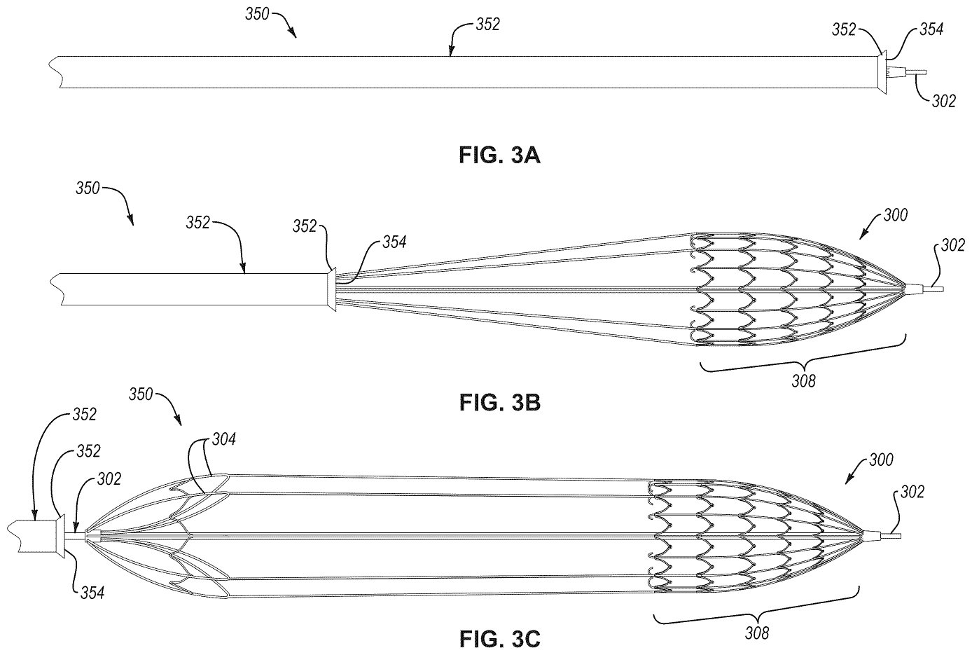

As previously discussed, the thrombectomy device 100 is configured to switch between a collapsed state when some of the components of the thrombectomy device 100 are positioned adjacent to the catheter 102 and a deployed state when at least a portion of these components are spaced from the catheter 102, as illustrated in FIGS. 3A-3C. In an embodiment, one or more components of the thrombectomy device 100 (e.g., the atraumatic fingers, the capture bag 108, etc.) may be formed of a superelastic material (e.g., nickel-titanium superelastic alloy). In such an embodiment, the superelastic material may allow at least these components of the thrombectomy device 100 to move away from the catheter 102 when in a relaxed state (i.e., no external compressive load is applied thereto by one or more external forces, such as the external containment sheath 352 of FIGS. 3A-3C) and the thrombectomy device 100 is in the deployed state while also allowing at least these components to be elastically deformed to be positioned proximate to the catheter 102.

In an embodiment, the thrombectomy device 100 may include at least one biasing element 146. The biasing element 146 is configured to move at least a portion of one or more components away from the catheter 102 when the thrombectomy device 100 is in the deployed state while allowing the components to move towards the catheter 102 when the thrombectomy device 100 is in the collapsed state. The thrombectomy device 100 may include the biasing element 146 when one or more components of the thrombectomy device 100 do or do not include a superelastic material. In an example, as shown, the biasing element 146 may include a curved beam attached to the proximal end of the atraumatic fingers 104 or the proximal region 114 and extend to a distal portion of the atraumatic fingers 104 or the coring ring 106. The curved beam may exhibit a concave curve relative to the atraumatic fingers 104 such that a portion of the curved beam presses against the catheter 102. The curved beam pressing against the catheter 102 may cause the curved beam to move the portions of the atraumatic fingers 104 not attached to the catheter 102 away from the catheter 102 when the thrombectomy device 100 is in the deployed state. In an example, the biasing element 146 may include another type of biasing element and/or may be attached to another component (e.g., the capture bag 108 or the linkages 110).

In an embodiment, as shown, the biasing element 146 is integrally formed with at least one of the atraumatic fingers 104 and/or at least one of the linkages 110. In such an embodiment, the biasing element 146 may be positioned to not contact the blood vessel during use. Instead, the biasing element 146 may be positioned between the atraumatic fingers 104 and/or the linkages 110 and the catheter 102. In an embodiment, the biasing element 146 is distinct and attached to at least one of the atraumatic fingers 104 and/or at least one of the linkages 110.

The atraumatic fingers 104, the coring ring 106, the capture bag 108, and the linkages 110 may be formed from any suitable material. In an example, the atraumatic fingers 104, the coring ring 106, the capture bag 108, and the linkages 110 may be independently selected to include at least one of a nickel-titanium superelastic alloy (e.g., nitinol) stainless steel, titanium, other suitable metals, other biocompatible materials, or combinations thereof. In an embodiment, one or more of the atraumatic fingers 104, the coring ring 106, the capture bag 108, or the linkages 110 may include the same material. In such an embodiment, these components of the thrombectomy device 100 may be integrally formed together which may facilitate manufacturing of the thrombectomy device 100. That said, the components of the thrombectomy device 100 including the same material may still be attached together using any of the techniques disclosed herein. In an embodiment, one or more of the atraumatic fingers 104, the coring ring 106, the capture bag 108, or the linkages 110 may include different materials. For example, the atraumatic fingers 104 may include a superelastic material that facilitate switching the thrombectomy device 100 from the collapsed state to the deployed state and the coring ring 106 may be formed of a material that can easily have the cutting edge 130 formed therein.

In an embodiment, one or more components of the thrombectomy device 100 may include at least one radiopaque marker or surface modification that is more easily detected by ultrasonic imaging technique or other medical imaging techniques than other components of the thrombectomy device 100. The radiopaque marker or surface modification may help view the thrombectomy device 100 during use. Examples of radiopaque markers includes forming a portion of the thrombectomy device 100 with platinum or rhodium bonded to, welded to, or alloyed with any of the components of the thrombectomy device 100. In an example, the radiopaque marker or surface modification may be formed in or near the proximal region 114, the coring ring 106, and/or the distal region 116 which may help indicate when the thrombectomy device 100 begins to engage with the thrombus, when the thrombectomy device 100 begins to cut and finishes cutting the thrombus, and when the thrombus reaches the capture bag 108, respectively.

In an embodiment, one or more components of the thrombectomy device 100 may include an anti-inflammatory or thrombolytic coating (e.g., silicon carbide) or drug disposed therein. For instance, the atraumatic fingers 104 (e.g., the exterior surfaces 122) may include the anti-inflammatory coating or drug disposed thereon to facilitate healing of the walls of the blood vessel in case operating the thrombectomy device 100 inadvertently damages. Other components of the thrombectomy device 100 that contact the walls of the blood vessel (e.g., the capture bag 108 or the linkages 110) may include the anti-inflammatory coating or drug disposed thereon. In an embodiment, the coring ring 106 may include an anti-inflammatory coating or drug in case the atraumatic fingers 104 fail to protect the walls of the blood vessel and the coring ring 106 inadvertently cuts or otherwise damages the walls of the blood vessel or the rubbing of the atraumatic fingers 104 cause some inflammation.

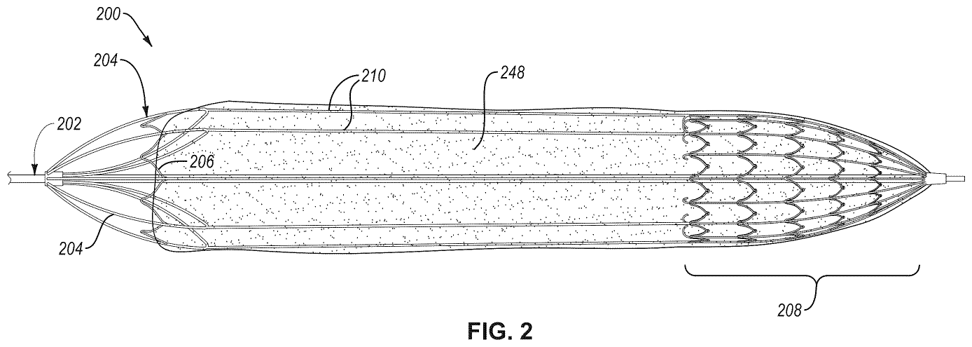

It is noted that the thrombectomy devices disclosed herein may exhibit structures other than the structure illustrated in FIGS. 1A and 1B. For example, FIG. 2 is a side view of a thrombectomy device 200, according to an embodiment. Except as otherwise disclosed herein, the thrombectomy device 200 is the same as or substantially similar to any of the thrombectomy devices disclosed herein. For example, the thrombectomy device 200 includes a catheter 202, a plurality of atraumatic fingers 204, a coring ring 206, and a capture bag 208.

The thrombectomy device 200 includes a porous cover 248. The porous cover 248 may be positioned external to and cover at least a portion of the thrombectomy device 200 that is distal to the coring ring 206. The porous cover 248 effectively reduces the size of any gaps that are present in the thrombectomy device 200 that is distal to the coring ring 206. As such, the porous cover 248 prevents portions of the thrombus from passing through gaps in the thrombectomy device 200 such that these portions of the thrombus are not removed from the blood vessel. In an example, the porous cover 248 may cover at least the capture bag 208 to ensure that the thrombus received by the capture bag 208 remains in the capture bag 208, for instance, even as the capture bag 208 switches to a collapsed state. In an example, the porous cover 248 may cover at least a portion of the linkages 210 thereby preventing portions of the thrombus from passing through the larger gaps of the linkages 210 before the thrombus is received by the capture bag 208.

The porous cover 248 may be formed of any suitable material. For example, the porous cover 248 may include a polytetrafluoroethylene (e.g., expanded polytetrafluoroethylene), polyether block amide, polyamide, polyimide, polyethylene terephthalate, fluorinated ethylene propylene, polyolefin, nylon, other thermoplastics, or any other suitable material. In an embodiment, the porous cover 248 may include an anti-inflammatory or thrombolytic drug disposed in the pores thereof. For example, during use, the porous cover 248 may contact and rub against the walls of the blood vessel and/or any portions of the thrombus that remain attached to the walls of the blood vessels. The drug(s) disposed in the porous cover 248 may facilitate healing of the blood vessel (e.g. heal damage caused by the porous cover 248 rubbing against the walls of the blood vessel) and/or help remove the remaining portions of the thrombus.

FIGS. 3A-3C are a side view of a thrombectomy system 350 including a thrombectomy device 300 and a method of switching the thrombectomy device 300 from a collapsed state to a deployed state, according to an embodiment. It is noted that the thrombectomy device 300 illustrated in FIGS. 3A-3C is the same as or substantially similar to the thrombectomy device 100 illustrated in FIGS. 1A and 1B. That said, the thrombectomy device 300 may include any of the thrombectomy devices disclosed herein.

The thrombectomy system 350 includes an external containment sheath 352. The external containment sheath 352 exhibits a generally hollow structure (e.g., tube structure). The external containment sheath 352 is configured to receive the thrombectomy device 300 when the thrombectomy device 300 is in the collapsed state. The external containment sheath 352 is configured to maintain the thrombectomy device 300 in the collapsed state by applying a compressive load that maintains at least some of the components of the thrombectomy device 300 adjacent to the catheter 302.

The thrombectomy device 300 may be switched from the collapsed state to the deployed state by moving the external containment sheath 352 in a proximal direction. For example, as shown in FIG. 3B, moving the external containment sheath 352 such that the external containment sheath 352 does not cover the capture bag 308 allows the capture bag 308 to move away from the catheter 302 such that the capture bag 308 is in the deployed state. Further movement of the external containment sheath 352 in a proximal direction, as shown in FIG. 3C, causes the external containment sheath 352 to no longer cover the atraumatic fingers 304 thereby allowing the atraumatic fingers 304 to move away from the catheter 302 such that the atraumatic fingers 304 are in the deployed state.

The thrombectomy device 300 may be switched from the deployed state to the collapsed state by moving the external containment sheath 352 in a distal direction. Moving the external containment sheath 352 in the distal direction may cause the atraumatic fingers 304 to reenter the interior of the external containment sheath 352 which, in turn, applies a compressive force to the atraumatic fingers 304 that causes the atraumatic fingers 304 to move towards the catheter 302. Similarly, moving the external containment sheath 352 in the distal direction may cause the capture bag 308 to reenter the interior of the external containment sheath 352 which, in turn, applies a compressive force to the capture bag 308 that causes the capture bag 308 to move towards to the catheter 302. The capture bag 308 entering the external containment sheath 352 may also cause a thrombus within the capture bag 308 to enter the external containment sheath 352. In some embodiment, a vacuum may be provided in the interior of the external containment sheath 352 to cause any portions of the thrombus that extrude out of the capture bag 308 as the capture bag 308 is compressed to still enter the external containment sheath 352.

In an embodiment, the external containment sheath 352 may include a flared, open distal end 354. The flared, open distal end 354 increases the diameter of the external containment sheath 352. The increased diameter of the external containment sheath 352 may facilitate collapsing the atraumatic fingers 304 and the capture bag 308 and may facilitate forcing the thrombus into the external containment sheath 352 when switching the thrombectomy device 300 from the deployed state to the collapsed state.

The thrombectomy system 350 may include one or more additional components that are not illustrated. In an example, the thrombectomy system 350 may include a handle that facilitates operation of the rest of the thrombectomy system 350. For instance, the handle may include an actuator that, when actuated, causes the external containment sheath 352 to move proximally and/or distally. In an example, the thrombectomy system 350 may include a guidewire (e.g., guidewire 456 illustrated in FIGS. 4A-4D) In an example, the thrombectomy system 350 may include an aspiration device. In an example, the thrombectomy system 350 may include components included in conventional thrombectomy systems.

FIGS. 4A-4D illustrate a method of using a thrombectomy system 450, according to an embodiment. Except as otherwise disclosed herein, the thrombectomy system 450 may be the same as or substantially similar to any of the thrombectomy systems disclosed herein. For example, the thrombectomy system 450 may include a thrombectomy device 400 and an external containment sheath 452. The thrombectomy device 400 may include a catheter 402, a plurality of atraumatic fingers 404, a coring ring 406, and a capture bag 408.

Referring to FIG. 4A, a guidewire 456 may be inserted into a blood vessel 458 effective to perforate the blood vessel 458 and advanced distally in and through the blood vessel 458 until the guidewire 456 extends through and past a thrombus 460. After the guidewire 456 is positioned, the thrombectomy system 450 may be positioned on the guidewire 456 such that the guidewire 456 is positioned in the cavity (e.g., cavity 118 shown in FIG. 1B) of the catheter 402. The thrombectomy device 400 may be displaced distally along the guidewire 456 (e.g., move distally relative to the guidewire 456) until at least the atraumatic fingers 404 (not illustrated in FIG. 4A, obscured by the external containment sheath 452) are positioned distally relative to the thrombus 460. For example, the thrombectomy system 450 (e.g., the thrombectomy device 400 and the external containment sheath 452) may be attached to a handle that is outside of the body. A proximal end of the guidewire 456 may be inserted into the distal end of the cavity of the catheter 402. A medical practitioner may then move the handle to displace the thrombectomy system 450 along the guidewire 456 until at least the atraumatic fingers 404 are positioned past the thrombus 460.

Referring to FIG. 4B, the external containment sheath 452 is moved proximally relative to the thrombectomy device 400 to switch the thrombectomy device 400 from the collapsed state (FIG. 4A) to the deployed state (FIG. 4B) after the thrombectomy device 400 is correctly positioned. In an example, the handle to which the thrombectomy system 450 is attached includes a first actuator (e.g., rotatable wheel). The medical practitioner may manipulate the first actuator (e.g., rotate the wheel), which may cause the distal end of the external containment sheath 452 to move towards the handle while maintaining the relative position of the thrombectomy device 400 relatively unchanged relative to the handle which, in turn, causes the external containment sheath 452 to move relative to the thrombectomy device 400. Such movement may switch the thrombectomy device 400 from the collapsed state to the deployed state. In an example, the handle to which the thrombectomy system 450 is attached includes a second actuator (e.g., rotatable wheel). The medical practitioner may manipulate the second actuator which may cause the distal end of the thrombectomy device 400 to move away from the handle while maintaining the relative position of the external containment sheath 452 relatively unchanged relative to the handle which, in turn, causes the thrombectomy device 400 to move relative to the external containment sheath 452.

When in the deployed state, the atraumatic fingers 404, the coring ring 406, the capture bag 408, and the linkages 410 are free to expand and move away from the catheter 402. At least the atraumatic fingers 404 may expand outwardly from the catheter 402 until a portion of the atraumatic fingers 404 abuts the wall 462 of the blood vessel 458 thereby allowing the atraumatic fingers 404 to protect the wall 462 from at least the coring ring 406. The capture bag 408 and the linkages 410 may also expand outwardly from the catheter 402 until the capture bag 408 and the linkages 410 contact the wall 462 which may ensure that the portions of the thrombus 460 cut by the coring ring 406 may flow through the space defined by the linkages 410 and into the volume defined by the capture bag 408.

Referring to FIG. 4C, the thrombectomy device 400 in the deployed state may be advanced proximally relative to the blood vessel 458. For example, the thrombectomy device 400 may be move proximally by at least one of moving the handle away from the patient or actuating a second actuator of the handle which, when manipulated, causes the thrombectomy device 400 (e.g., at least the atraumatic fingers 404, the coring ring 406, the capture bag 408, and the linkages 410) to move towards the handle. Such movement of the thrombectomy device 400 allows the coring ring 406 to engage with the thrombus 460 and to cut the thrombus 460 from the wall 462 of the blood vessel 458. The atraumatic fingers 404 form a barrier between the coring ring 406 and the wall 462 such that the coring ring 406 only cuts the thrombus 460 with minimal damage caused to the wall 462.

Referring to FIG. 4D, after the coring ring 406 cuts the thrombus 460, the cut thrombus 460 may move distally relative to the thrombectomy device 400, through the space defined by the linkages 410 and into the capture bag 408. The thrombus may move distally relative to the thrombectomy device 400, for example, by moving the thrombectomy device 400 proximally relative to the blood vessel 458 (e.g., using any of the techniques disclosed above).

Once the thrombus 460 is in the capture bag 408, the thrombectomy device 400 may be removed from the blood vessel 458. In an embodiment, not shown, removing the thrombectomy device 400 from the blood vessel 458 may include moving the thrombectomy device 400 into the external containment sheath 452 to switch the thrombectomy device 400 from the deployed state to the collapsed state. Moving the thrombectomy device 400 into the external containment sheath 452 also causes the cut thrombus 460 that is within the capture bag 408 to also move into the external containment sheath 452. The thrombectomy device 400 may be moved into the external containment sheath 452 using any suitable technique. In an example, the first actuator of the handle may be manipulated by the medical practitioner such that the distal end of the external containment sheath 452 moves away from the handle while keeping the relative position of the thrombectomy device 400 relative to the handle substantially constant. Such movement of the external containment sheath 452 may cause the external containment sheath 452 to collapse the thrombectomy device 400 and receive the thrombectomy device 400 into an internal cavity of the external containment sheath 452. Receiving the thrombectomy device 400 into the internal cavity of the external containment sheath 452 also causes the thrombus 460 to be received into the internal cavity. A suction may be provided to the internal cavity of the external containment sheath 452 to ensure that all or substantially all of the thrombus is received into the internal cavity. In an example, the second actuator of the handle may be manipulated by the medical practitioner which causes the thrombectomy device 400 to displace proximally into the external containment sheath 452 while the position of the external containment sheath 452 relative to the handle remains substantially unchanged. In an example, the thrombectomy device 400 may be moved into the external containment sheath 452 responsive to manipulation of both the first and second actuators of the handle.

It is noted that the thrombectomy system 450 may be displaced or otherwise move relative to the guidewire 456, the thrombectomy system 450 may be positioned in and move relative to the blood vessel 458, and the thrombectomy device 400 may be displaced or otherwise move relative to the external containment sheath 452 using any other suitable technique, as known in the art.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. A thrombectomy device, comprising:
a catheter having a proximal region and a distal region;
a plurality of atraumatic fingers extending from the proximal region, the plurality of atraumatic fingers collectively defining an outer periphery of the plurality of atraumatic fingers;
a coring ring attached to or integrally formed with at least some of the plurality of atraumatic fingers, at least a portion of the coring ring positioned within the outer periphery of the plurality of atraumatic fingers;
a capture bag extending from the distal region;
wherein the thrombectomy device is switchable between a compact state and a deployed state, wherein the plurality of atraumatic fingers, the coring ring, and the capture bag are adjacent to the catheter when the thrombectomy device is in the compact state and at least partially spaced from the catheter when the thrombectomy device is in the deployed state.

2. The thrombectomy device of claim 1, wherein the plurality of atraumatic fingers includes a nickel-titanium superelastic alloy.

3. The thrombectomy device of claim 1, wherein the plurality of atraumatic fingers exhibit a relaxed shape when no compressive load is applied thereto, and wherein the relaxed shape of the plurality of atraumatic fingers causes the thrombectomy device to switch from the collapsed state to the deployed state when no compressive load is applied to the plurality of atraumatic fingers.

4. The thrombectomy device of claim 1, wherein all of the coring ring is positioned within the outer periphery of the plurality of atraumatic fingers.

5. The thrombectomy device of claim 1, wherein the coring ring is attached to an interior surface of the plurality of atraumatic fingers, the interior surface of the plurality of atraumatic fingers is a surface of the plurality of atraumatic fingers closest to the catheter.

6. The thrombectomy device of claim 1, wherein the coring ring exhibits a zigzag structure including a plurality of peaks pointing in a proximal direction and a plurality of valleys pointing in a distal direction.

7. The thrombectomy device of claim 6, wherein portions of the coring ring defining at least some of the plurality of valleys are attached or integrally formed with the plurality of atraumatic fingers.

8. The thrombectomy device of claim 1, wherein the coring ring exhibits a generally circular cross-sectional shape.

9. The thrombectomy device of claim 1, wherein the coring ring includes one or more bending regions that are configured to bend when the thrombectomy device is in the collapsed state.

10. The thrombectomy device of claim 1, wherein the coring ring includes a cutting edge on a proximal side of the coring ring.

11. The thrombectomy device of claim 1, wherein the coring ring includes steel.

12. The thrombectomy device of claim 1, further comprising at least one biasing element configured to switch the thrombectomy device from the compact state to the deployed state.

13. The thrombectomy device of claim 1, further comprising a plurality of linkages extending between at least some of the plurality of atraumatic fingers and the capture bag.

14. The thrombectomy device of claim 13, wherein the plurality of linkages are connected to the plurality of atraumatic fingers at one end and the capture bag at the other end.

15. The thrombectomy device of claim 1, further comprising a porous bag extending at least partially around the capture bag or a plurality of linkages extending from the plurality of atraumatic fingers to the capture bag.

16. The thrombectomy device of claim 15, wherein the porous bag is embedded with at least one of a thrombolytic drug or an anti-inflammatory drug.

17. The thrombectomy device of claim 15, wherein the porous bag includes expanded polytetrafluoroethylene.

18. A thrombectomy system, comprising:
a thrombectomy device including:
a catheter having a proximal region and a distal region;
a plurality of atraumatic fingers extending from the proximal region, the plurality of atraumatic fingers collectively defining an outer periphery of the plurality of atraumatic fingers;
a coring ring attached to or integrally formed with at least some of the plurality of atraumatic fingers, at least a portion of the coring ring positioned within the outer periphery of the plurality of atraumatic fingers;
a capture bag extending from the distal region;
wherein the thrombectomy device is switchable between a compact state and a deployed state, wherein the plurality of atraumatic fingers, the coring ring, and the capture bag are adjacent to the catheter when the thrombectomy device is in the compact state and at least partially spaced from the catheter when the thrombectomy device is in the deployed state
an external containment sheath moveably disposed on at least a portion of the thrombectomy device;
wherein the thrombectomy device is in the collapsed state when the external containment sheath is disposed over the plurality of atraumatic fingers, the coring ring, and the capture bag; and
wherein displacing the thrombectomy device distally out of the external containment sheath a sufficient distance switches the thrombectomy device from the collapsed state to the deployed state.

19. The thrombectomy system of claim 18, wherein the external containment sheath includes a flared, open distal end.

20. A method of using a thrombectomy device, the method comprising:
advancing the thrombectomy device in a collapsed state through a thrombus such that a plurality of atraumatic fingers of the thrombectomy device is positioned distal to the thrombus, the thrombectomy device including:
a catheter having a proximal region and a distal region;
the plurality of atraumatic fingers extending from the proximal region, the plurality of atraumatic fingers collectively defining an outer periphery of the plurality of atraumatic fingers;
a coring ring attached to or integrally formed with at least some of the plurality of atraumatic fingers, at least a portion of the coring ring positioned within the outer periphery of the plurality of atraumatic fingers;
a capture bag extending from the distal region;
wherein the plurality of atraumatic fingers, the coring ring, and the capture bag are adjacent to the catheter when in the collapsed state;
moving an external containment sheath proximally for a sufficient distance to switch the thrombectomy device from the collapsed state to a deployed state, wherein the coring ring, and the capture bag are at least partially spaced from the catheter when in the deployed state; and
advancing the thrombectomy device proximally to cut the thrombus from a wall of a blood vessel and disposed the cut thrombus in the capture bag.
